(19) United States
(12) Patent Application Publication
(10) Pub. No.: US 2010/0144820 A1
(43) Pub. Date: Jun. 10, 2010

(54) THERAPEUTIC HPPH DOSAGE FOR PDT

(75) Inventors: Ravindra K. Pandey, Williamsville, NY (US); Thomas J. Dougherty, Grand Island, NY (US); William R. Potter, Amherst, NY (US)

Correspondence Address: MICHAEL L. DUNN, SIMPSON & SIMPSON, PLLC, 5555 MAIN STREET, WILLIAMSVILLE, NY, 14221, US

(73) Assignee: Health Research, Inc., Buffalo, NY (US)

(21) Appl. No.: 12/448,661

(22) PCT Filed: Sep. 27, 2007
(86) PCT No.: PCT/US2007/020818

§ 371 Date: Feb. 5, 2010

Related U.S. Application Data
(60) Provisional application No. 60/879,474, filed on Jan. 9, 2007.
(60) Provisional application No. 60/879,435, filed on Jan. 9, 2007.
(60) Provisional application No. 60/967,652, filed on Sep. 6, 2007.

(51) Int. Cl. A61K 31/409; C07D 487/22; A61P 35/00

(52) U.S. Cl. 514/410; 540/145

(57) ABSTRACT

A method for treating cancer and other hyperproliferative tissues in humans that can be exposed to light comprising injection of HPPH at the equivalent to a dose of 0.05 to 0.11 mg/kg of body weight 24 hours post injection and exposing the tumor or other hyperproliferative tissue to 665±10 nm of light at 50 to 200 Joules/cm².

### CROSS REFERENCE TO RELATED PATENT APPLICATIONS

This application claims priority from U.S. Provisional Application Nos. 60,967,652, filed 6 Sep. 2007; 60/879,435, filed 9 Jan. 2007; and 60/879,474, filed 9 Jan. 2007.

### STATEMENT REGARDING FEDERALLY SPONSORED RESEARCH

This invention was made with funding from the national Institute of Health Grant Numbers NIH (IR21 CA109914-01 and CA 55792). The United States Government may have certain rights in this invention.

### BACKGROUND OF THE INVENTION

Photodynamic therapy (PDT) is believed to exploit the biological consequences of localized oxidative damage inflicted by photodynamic processes. Three critical elements required for initial photodynamic processes to occur are: a photosensitizer, light at the photosensitizer-specific absorption frequency or wavelength, and oxygen. The light at the required wavelength is believed to trigger singlet oxygen production to destroy tissue in which it is concentrated.

Tetrapyrrolic photosensitizers, such as the photosensitizer porfimer sodium, sold under the trademark PHOTOFRIN™ (The only FDA approved Photosensitizer in the United States), and HPPH, concentrate well in most tumor tissue.

For these reasons, for the last several years porphyrin-based compounds have been used for the treatment of cancer by photodynamic therapy (PDT). The concentration of certain porphyrins and related tetrapyrrolic systems is higher in malignant tumors than in most normal tissues and that has been one of the main reason for using these molecules as photosensitizers. Some tetrapyrrole-based compounds, e.g. PHOTOFRIN®, (porfimer sodium, described in the background of U.S. Reissue Pat. No. RE38,094 incorporated herein by reference) have been effective in a wide variety of malignancies, including skin, lung, bladder, head and neck and esophagus and other hyperproliferive tissues such as Barrett's Esophagus and macular degeneration. There have, however been associated problems with their use including skin phototoxicity, normal tissue damage, insufficient depth of penetration and a high percentage of esophageal strictures. The precise mechanism(s) of PDT are unknown; however, in vivo animal data suggests that both direct cell killing and loss of tumor vascular function play a significant role. A new and well tested tetrapyrrolic compound is the 2-(1-hexyloxy)-2-ethyl-derivative of pyropheophorbide-a (HPPH). HPPH, as used herein, means the 2-(1-hexyloxy)-2-ethyl-derivative of pyropheophorbide-a in both its free acid and ester and salt forms. This compound is tumor-avid and has undergone Phase I/II human clinical trials at the Roswell Park Cancer Institute in Buffalo, N.Y. Initial dosages for this compound were selected based upon ten percent of the lowest toxic dose in surfactant containing liquid media in dogs. The lowest toxic dose for HPPH in 0.1% Tween 80® (polysorbate 80) surfactant solution is about 1.5 mg/kg. At these levels neuropathy is common in test animals. The toxicity is due to surfactant rather than the HPPH. Such doses have thus been about 0.15 mg/kg of body weight or about 6 mg/m² of body surface area based upon an average 70 kg man having a two square meter surface area. Based upon clinical trials with prior photosensitizers, dosages less than ten percent of the lowest toxic dose have generally not been found to be effective. Further, light energy to activate HPPH has similarly been based upon prior research with other photosensitizers, i.e. about 135 to about 283 Joules/cm². This was believed to be essentially confirmed for HPPH by studies in mice, e.g. as presented in Table 1 of U.S. Pat. No. RE 38,094 showing no or extremely little long term tumor response at dosages of 0.05 to 0.3 mg/kg at energy of 135 Joules/cm².

While HPPH has much less prolonged phototoxicity over time than other photosensitizers and can be used with much less normal tissue damage in treating tumors and other hyperproliferative tissue; nevertheless, erythema and other damage can occur. It would be desirable to obtain high tumor response without systemic toxic effects and phototoxicity.

### BRIEF DESCRIPTION OF THE INVENTION

In accordance with the invention it has been unexpectedly discovered that in humans many cancers and other hyperproliferative tissues can be effectively treated by injection of HPPH at the equivalent to a dose of 0.05 to 0.11 mg/kg of body weight 24 hours post injection and exposed to 665±10 nm of light at a total delivered light dose of 50 to 200 joules/cm². The lower limit is preferably 75 almost preferably 100 Joules/cm² and the upper limit is preferably 150 Joules/cm².

HPPH, i.e. 2-(1-hexyloxy)-2-ethyl-derivative of pyropheophorbide-a, has the following formula:

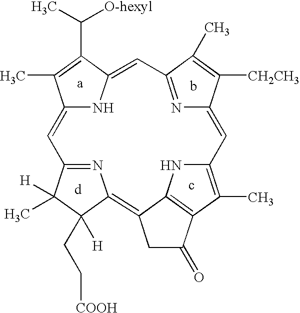

and includes the salts thereof and may be prepared as set forth in U.S. Pat. Nos. 5,198,460 and 5,314,905 reissued as RE39094 and RE38994 respectively, all of which are incorporated herein by reference.

### DETAILED DESCRIPTION OF THE INVENTION

The HPPH is preferably injected as a part of a composition comprising 0.5 to 1.5 mg/ml HPPH, 0.05 to 0.15 wt. percent surfactant having a hydrophilic-lipophilic balance HLB of 14 to 16, 1 to 3 wt. percent ethanol and 3 to 8 wt. percent monosaccharide, preferably glucose, with the balance being water. Preferred surfactants are polysorbate 80 and sucrose ester, e.g. sucrose laurate or sucrose stearate.

For most applications, the preferred dose of HPPH is from 0.07 to 0.1 mg/kg of body weight and the preferred light dose is from 75 to 150 Joules/cm²

Table 1 gives results of a study of Photodynamic Therapy for the treatment of basal cell carcinoma using 2-(1-hexyloxyethyl)-2-devinyl pyropheophorbide-a (HPPH).

TABLE 1
Results of treatment of basal cell carcinoma with HPPH at various conditions
Treatment Energy

| | | | | | | | Adverse |
|---|---|---|---|---|---|---|---|
| | 50 J/cm² | 100 J/cm² | 150 J/cm² | 200 J/cm² | 150 J/cm² | 200 J/cm² | Effects |
| Dose | @24 hr | @24 hr | @24 hr* | @24 hr* | @48 hr | @48 hr | Noted |
| 0.05 mg/kg* | | | Complete | | | | 1 |
| 0.05 mg/kg* | | | Complete | | | | 1 |
| 0.05 mg/kg* | | | Complete | | | | 1 |
| 0.05 mg/kg* | | | | Complete | | | 1 |
| 0.05 mg/kg* | | | | Complete | | | 1 |
| 0.05 mg/kg* | | | | Complete | | | 1 |
| 0.07 mg/kg | Fail | | | | | | 1 |
| 0.07 mg/kg | Partial | | | | | | 1 |
| 0.07 mg/kg | Complete | | | | | | 1 |
| 0.07 mg/kg | | Complete | | | | | 1 |
| 0.07 mg/kg | | Complete | | | | | 1 |
| 0.07 mg/kg | | Complete | | | | | 1 |
| 0.07 mg/kg* | | | Complete | | | | 1 |
| 0.07 mg/kg* | | | Complete | | | | 1 |
| 0.07 mg/kg* | | | Complete | | | | 1 |
| 0.07 mg/kg* | | | | Complete | | | 1 |
| 0.07 mg/kg* | | | | Complete | | | 1 |
| 0.07 mg/kg* | | | | Complete | | | 1 |
| 0.07 mg/kg | | | | | Complete | | 1 |
| 0.07 mg/kg | | | | | Complete | | 1 |
| 0.07 mg/kg | | | | | Complete | | 1 |
| 0.07 mg/kg | | | | | | Complete | 1 |
| 0.07 mg/kg | | | | | | Complete | 1 |
| 0.07 mg/kg | | | | | | Complete | 1 |
| 0.09 mg/kg | Partial | | | | | | 1 |
| 0.09 mg/kg | Partial | | | | | | 1 |
| 0.09 mg/kg | Complete | | | | | | 1 |
| 0.09 mg/kg | | Complete | | | | | 1 |
| 0.09 mg/kg | | Partial | | | | | 1 |
| 0.09 mg/kg | | Complete | | | | | 1 |
| 0.09 mg/kg | | | | | Complete | | 1 |
| 0.09 mg/kg | | | | | Complete | | 1 |
| 0.09 mg/kg | | | | | Complete | | 1 |
| 0.09 mg/kg | | | | | | Complete | 1 |
| 0.09 mg/kg | | | | | | Complete | 1 |
| 0.09 mg/kg | | | | | | Complete | 1 |
| 0.12 mg/kg* | | control | | | | | 3 |
| 0.12 mg/kg* | | control | | | | | 2 |
| 0.17 mg/kg | | | | | control | | 3-6 |
| 0.17 mg/kg | | | | | control | | 2-6 |

*= interpolated from 48 hr data based upon 30% elimination of drug between 24 and 48 hrs.

All dose data converted from actual drug dose in milligrams per square meter of surface area of patient assuming a 70 kg patient having a 2 m² surface area.

Adverse effects for normal skin tissue when exposed to 133 Joules/cm² (approximately equivalent to 30 minutes of midday sun exposure in Northeastern United States): 1=faint erythema; 2=minimal erythema with sharp borders; 3=pronounced erythema without edema; 6- more than 10% of toxic dose causing peripheral neuropathy in animals.

For the FDA approved PHOTOFRIN® porphyrin photosensitizer, very serious erythema and necrosis has been reported at 2 mg/kg dose (the lowest reasonable effective dose) upon exposure to only 9 Joules/cm² of activating light and serious erythema has also been reported for FOSCAN™ ALA-induced protoporphyrin photosensitizer upon only 30 minute sun exposure a full week after injection.

TABLE 2
Interluminal HPPH-PDT Treatment of Bronchiogenic Carcinoma
665 ± 5 nm Light Energy 48 HRS Post Injection

| | 75 J/cm | 75 J/cm | 85 J/cm | 85 J/cm |
|---|---|---|---|---|
| | Short | Long | Short | Long |
| | Term | Term | Term | Term |
| Dose | Response | Response | Response | Response |
| 0.1 mg/kg | CR | CR | | |
| 0.1 mg/kg | CR | Rec | | |
| 0.1 mg/kg | | | CR | CR |
| 0.1 mg/kg | | | CR | Rec |

Dosages are in Joules per cm length of light delivering optical fiber. Four hundred Joules per cm of diffusion fiber, the usual fiber dose for interluminal cancer, e.g. lung and esophagus is approximately equivalent to 50 Joules/cm² at the bronchial lumen No observable systemic or phototoxic side effects.

Palliative Treatment of Advanced Obstructive Endobronchial Lung Cancer For relief in breathing only. Long term responses not expected due to most patients having wide spread disease outside of the area treated by HPPH in the Bronchus. All HPPH doses were 0.1 mg/kg with exposure of 100 to 140 Joules per cm of diffusion fiber at 665±5 nm Light Energy 48 HRS Post Injection.

TABLE 3

| | 100 J/cm | 110 J/cm | 130 J/cm | 140 J/cm |
|---|---|---|---|---|
| 2 Month | 1 CR/2 PR | 3 PR | 2 CR/1 PR | 1 CR |
| FU | | | | |
| Long Term | 1 disease | 3 Lost | 2 new | 1 new |
| FU | free/ | | growth/ | growth |
| | 3Lost | | 1 lost | |

In all cases at least partial palliative response occurred, i.e. improved ability to breathe and surprisingly one was disease free upon long term follow up. No observable systemic or phototoxic side effects.

Use of HPPH Photodynamic Therapy for treatment of high grade dysplasia-carcinoma in Barrett's Esophagus. All energy was at 175 Joules/cm at 665±5 nm Light Energy 48 HRS Post Injection. This was a single treatment. Based on studies with PHOTOFRIN®, it is expected that three treatments, instead of just one, would result in about an 77% cure rate.

TABLE 4

Dose

| | 0.075 mg/kg | 0.1 mg/kg |
|---|---|---|
| 2 mo/FU | 3 PR/2 CR | 4 PR/3 CR |
| Long | 3 PR/2 CR | 1 Rec |
| Term/FU | | 4 PR/1 CR |

As can be seen from the above results, good tumor response can be obtained using HPPH at an unexpectedly low dose of 0.05 to 0.11 mg/kg, preferably 0.6 to 0.1 mg/kg, of body weight in humans, compared to the required PHOTOFIN® dose of 2 mg/kg of body weight. This is contrary to what would be expected from studies in mice as previously described.

The lower dose at relatively low energy permits good treatment with reduced risk of systemic toxicity and little or no phototoxicity.

What is claimed is:

1. A method for treating cancer and other hyperproliferative tissues in humans that can be exposed to light comprising injection of HPPH at the equivalent to a dose of 0.05 to 0.11 mg/kg of body weight 24 hours post injection and exposing the tumor or other hyperproliferative tissue to 665±10 nm of light at 50 to 200 Joules/cm².

2. The method of claim 1 where the light is 75 to 150 Joules/cm²

3. The method of claim 1 where the tissue is basal cell carcinoma.

4. The method of claim 1 where the tissue is advanced lung cancer tissue.

5. The method of claim 1 where the tissue is early stage lung cancer tissue.

6. The method of claim 1 where the tissue is high grade dysplasia carcinoma in Barrett's Esophagus.

7. The method of claim 1 where the tissue is advanced obstructing esopageal cancer.

8. The method of claim 1 where the tissue is head and neck cancer tissue.

9. HPPH for use in treating cancer and other hyperproliferative tissues in humans that can be exposed to light comprising injection of HPPH at the equivalent to a dose of 0.05 to 0.11 mg/kg of body weight 24 hours post injection and exposing the tumor or other hyperproliferative tissue to 665±10 nm of light at 100 to 200 joules/cm².

10. HPPH as claimed in claim 9 where the light is 75 to 150 Joules/cm²

11. The method of claim 1 where the HPPH is injected in a composition comprising 0.5 to 1.5 mg/kg HPPH, 0.05 to 0.15 weight percent surfactant having an HLB of 14 to 16, 1 to 3 weight percent ethanol and 3 to 8 weight percent monosaccharide with the balance being water.

12. The method of claim 11 where the monosaccharide is glucose and the surfactant is polysorbate 80.

13. The method of claim 11 where the surfactant is a sucrose ester.
